(19) United States
(12) Patent Application Publication
(10) Pub. No.: US 2026/0060988 A1
(43) Pub. Date: Mar. 5, 2026

(54) TRANSIENT LOW-DOSE METHOTREXATE FOR TOLERANCE TO ALLOIMMUNITY

(71) Applicant: Vitalant, Scottsdale, AZ (US)

(72) Inventors: Johnson Q. Tran, San Jose, CA (US); Rachael Jackman, San Francisco, CA (US)

(21) Appl. No.: 19/318,536

(22) Filed: Sep. 4, 2025

Related U.S. Application Data
(60) Provisional application No. 63/691,106, filed on Sep. 5, 2024.

(51) Int. Cl. A61K 31/519; A61K 35/19; A61P 37/06

(52) U.S. Cl. CPC A61K 31/519; A61K 35/19; A61P 37/06

(57) ABSTRACT

Methods and compositions for inducing immunotolerance in a subject to allogeneic transfusions and transplantations are provided. Further provided are methods for preventing or reducing alloimmunization and/or alloimmune sequelae.

### CROSS REFERENCE TO RELATED APPLICATION

The present application claims the benefit of U.S. Provisional Patent Application No. 63/691,106, entitled, “TRANSIENT LOW-DOSE METHOTREXATE FOR TOLERANCE TO ALLOIMMUNITY” filed Sep. 5, 2025. The contents of each of the aforementioned applications is hereby incorporated by reference in its entirety.

### FIELD

The disclosure relates generally to immunology, and more specifically to methods of preventing or reducing alloimmunization or immune responses, and applications thereof in allogeneic transfusions and transplantations.

### BACKGROUND

Allogeneic immune responses to platelet transfusion antigens can lead to serious complications, including platelet refractoriness or rejection of solid organs or hematopoietic stem cell transplants (HSCTs). This immune response often targets major histocompatibility complex (MHC) antigens on platelets and residual white blood cells (WBCs) in transfused products. Pre-existing anti-MHC antibodies are particularly problematic, increasing the risk of HSCT failure by 7.47-fold. Although modern leukoreduction techniques significantly reduce the risk of alloimmunization, they do not eliminate it entirely. Recipients of multiple transfusions are inherently higher risk of alloimmunization due to increased frequency of exposure to platelet antigens, making them prime candidates for preventative measures.

Current clinical practice manages alloimmunization by matching donor and recipient platelets. However, this approach is only viable for documented cases of alloimmunization-mediated refractoriness due to the challenges of finding perfect matches, the short shelf-life of platelets, heterogeneity of alloantigens, and the high costs of testing. Currently, there is no approved clinical intervention to prevent platelet alloimmunization. Alternative strategies, such as pathogen reduction by ultraviolet irradiation, have shown promise in animal studies but have not yet been successful for tolerance induction in clinical settings. Immunosuppressive agents like cyclosporine A and CTLA-Ig have also been evaluated, but their significant immunosuppressive effects limit their use. Given the persistent clinical concern of alloimmunization, especially among high-risk patients who require chronic transfusions, there is a critical need for safe and effective strategies to induce immune tolerance.

Therefore, there is an unmet need for developing methods for inducing long-term immune tolerance, prevent alloimmunization, mitigate alloimmune sequelae, and prevent subsequent transplant rejection and platelet refractoriness in patients undergoing or planning to undergo transfusion, organ transplant, or stem cell transplant.

### SUMMARY

In one aspect, the present disclosure encompasses a method of preventing or reducing alloimmunization and/or alloimmune sequelae in a subject in need thereof, comprising: administering a first dose of methotrexate (MTX) concurrently with a first allogeneic transfusion, thereby preventing or reducing alloimmunization and/or alloimmune sequelae in the subject. The method can further comprise administering a second dose of MTX. In one aspect, the method further comprises administering a third dose of MTX.

The disclosure further provides a method of inducing a long-term immune tolerance to prevent or reduce alloimmunization in a subject in need thereof, comprising: administering a first dose of MTX concurrently with a first allogeneic transfusion; administering a second dose of MTX, and administering a third dose of MTX, thereby inducing long-term immune tolerance in the subject.

A method of reducing risk of bone marrow transplant rejection in a subject in need thereof is further provided in the disclosure. The method comprises administering a first dose of MTX concurrently with a first allogeneic platelet transfusion in a subject planning to undergo or have undergone bone marrow transplant, wherein administering MTX concurrently with platelet transfusion reduces the risk of bone marrow transplant rejection. In one aspect, the method further comprising administering a second dose of MTX. In yet another aspect, the method further comprising administering a third dose of MTX.

In some aspects, a method of preventing or reducing alloimmunization and/or alloimmune sequelae in a subject in need thereof, comprising, administering a first dose of MTX concurrently with a first allogeneic non-leukoreduced platelet transfusion; administering a second dose of MTX; and administering a third dose of MTX thereby preventing or reducing alloimmunization and/or alloimmune sequelae in the subject is further provided.

In certain aspects, the first, second and third dose of MTX is about 0.1 mg/kg to about 7 mg/kg. The first, second, and third doses may be administered as a daily dose. The disclosed daily dose may comprise administering a daily dose of MTX on 4^{th}, 5^{th}, 6^{th}, 7^{th}, 8^{th}, 9^{th}, 10^{th}, or 11^{th} day after administration of the first dose. In one aspect, the doses may be administered on consecutive days or non-consecutive days.

In some aspects, the methods (a) enhances the level of one or more of regulatory B cells, regulatory T cells, IL-10, and TGF-beta; (b) reduces the level of one or more of TNF-alpha, IFN-gamma, IL-2, IL-12, IL-4, IL-5, and IL-13; and/or (c) reduces the total alloantibody level or skews the alloantibody response towards isotypes that are less inflammatory or more immunotolerant, in the subject. In another aspect, the method prevents or reduces alloimmunization in the subject for a subsequent transfusion. In one aspect, the method further comprises a second transfusion.

The methods disclosed herein further comprises transfusion of allogeneic platelets. The transfusion comprises allogeneic leukoreduced platelets or non-leukoreduced platelets.

In one aspect, the methods prevent or reduce alloimmunization in the subject for at least 12 weeks.

In another aspect, the methods disclosed herein further comprising administering a dose of TLD-MTX before the administration of the first platelet transfusion.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows that transient low-dose methotrexate (TLD-MTX) diminished persistent alloantibodies after a single platelet transfusion. Class I MHC alloantibody screen of serum collected over time from three independent experiments (N=8−9). B6 recipient mice given a single BALB/c donor allogeneic non-luekoreduced platelet transfusion alone (NLR) or concurrently with TLD-MTX (NLR+TLD-MTX). Untreated or TLD-MTX only groups served as control. All median fluorescence intensity (MFI) values normalized by average MFI of untreated group. Mean and SEM shown. 2-way ANOVA with Turkey's post-hoc test. *p<0.05 comparing NLR vs NLR+ TLD-MTX groups.

FIG. 2 shows that TLD-MTX reduced alloantibodies after secondary platelet transfusion. Class I MHC alloantibody screen of serum from three independent experiments (N=9). B6 recipient mice from FIG. 1 were given a second allogeneic platelet transfusion after 12 weeks. All MFI values normalized by dividing with average MFI of untreated group. Tick and shaded region represent highest primary response from the NLR group from FIG. 1. Mean and SEM shown. 2-way ANOVA with Tukey's post-hoc test showed no significance for NLR vs NLR +TLD-MTX.

FIG. 3A-3D show Allo-B cell responses after a single allogeneic transfusion. FIG. 3A shows gating strategy to identify allogeneic-specific B cells (allo-B cells) with I-A^{d} MHC tetramers. Gating strategy first selected for singlet, live, lymphocytes (not shown). Then B cells (B220⁺), syngeneic decoy⁻ events. MHC tetramers (Tet) conjugated to fluorophores APC and PE were used to identify double positive events (allo-B cells). FIGS. 3A-3D show recipient B6 allo-B cells (FIG. 3B) expand, (FIG. 3C) are activated (CD86⁺), and (FIG. 3D) differentiate into germinal center B cells (GC, CD95₊/CD38⁻) in the spleen 10 days after a single non-leukoreduced (NLR) allogeneic platelet transfusion from BALB/c donors. Data pooled from three independent experiments (N=8-9). Mean and SEM shown. T-test **p<0.01, ***p<0.001.

FIG. 4A-4D show experimental setup. FIG. 4A shows platelet allogeneic transfusion (Allo Tx) model. FIG. 4B shows experimental groups for Aim 1. 1=primary transfusion. NLR=leukoreduced. LR=Leukoreduced. TLD-MTX=transient low-dose methotrexate. FIG. 4C shows additional experimental groups. FIG. 4D shows the primary allogeneic platelet transfusion is given concurrently with a single cycle of TLD-MTX. Weekly platelet transfusions are administered thereafter up until 12 weeks. IP=intraperitoneal.

FIG. 5 shows that allo-B cell B_{Regs} elevated after a single allogeneic transfusion. Allo-B cells were first gated as described in FIG. 3A. Then the subset of B10 B_{Regs} (CD1d⁺/CD5⁺) of allo-B cells in the spleen were examined. Allo-B cell B_{Regs} were significantly enriched 10 days after a single allogeneic transfusion. Allo-B cells were identified with H-2D^{d} MHC tetramers. Data pooled from three independent experiments (N=8-9). Mean and SEM shown. T-test *p<0.05.

FIG. 6A-6B show T regulatory cells (T_{Regs}) after a single allogeneic transfusion. T_{Regs} in the bulk T cell population. FIG. 6A shows CD4⁺/FoxP3⁺/CD25⁺ T cells (T_{Regs}) and FIG. 6B shows the subset of CTLA-4⁺/TGF-beta⁺ T_{Regs} from spleen over time after a single allogeneic transfusion (NLR). The y-axis show number of cells x 10³. 2-way ANOVA with Tukey's post-hoc test. N=10. *p<0.05, ***p<0.001, **** p<0.0001.

FIG. 7 shows the cytokine profile after an allogeneic transfusion. Two weeks after a single allogeneic transfusion, splenocytes were harvested and cultured alone (x) or with mitomycin C treated lymphocytes from donor (O) for 48 hours. Cytokines were measured in supernatants by multiplexing methods (N=5). T-test *p<0.05, **p<0.01, ***p<0.001, and ****p<00.0001.

FIG. 8 shows total alloantibodies after a single and multiple allogeneic transfusions. Total alloantibodies after a single allogeneic NLR platelet transfusion (left) and after multiple weekly transfusions with NLR or LR platelets (right). Normalized to untreated. T-test *p<00.0001.

FIG. 9 shows preliminary safety data shows TLD-MTX does not negatively impact platelets. Left panel shows platelet counts of TLD-MTX alone are enriched then stabilizes relative to Untreated and right panel shows platelet counts fall below normal values in mice treated with chemotherapeutic agents (Chemo; cytarabine and doxorubicin) or irradiation (800 cGy). Dotted lines and gray shade represent range of normal platelet values in mice. Shown are standard deviations (SD). N=5-10 per group.

FIG. 10 shows platelet refractoriness due to transferred serum alloantibodies from multiply transfused recipients with allogeneic platelets. Pooled serum from recipients that received weekly allogeneic platelet were diluted as indicated and 400 μl transferred ip into naive recipients. GFP+ donor-type platelets were transfused (Tx) to enable tracking of platelet recovery. Delivery of GFP+ donor-type platelets alone or with PBS served as controls. % recovery of GFP+ circulating platelets was measured at 24 hours by flow. Recovery was based on # of platelets transfused and estimated total circulating platelets by weight. *p<0.001.

FIG. 11 shows a schematic that summarizes the proposed TLD-MTX tolerance induction to prevent alloimmune sequelae. On the left, a single allogeneic platelet transfusion leads to unregulated allo-B cell activation, formation of plasma cells that produce inflammatory alloantibodies IgG2b and IgG2c and leads to transplant rejection and platelet refractoriness. However, in the presence of TLD-MTX on the right, induction of T_{Regs} and B_{Regs} suppress allo-B cell activity and lead to regulatory IgG1 alloantibodies which are permissive to platelet and transplant survival.

### DETAILED DESCRIPTION

The present disclosure describes methods and compositions to induce tolerance, reduce risk of alloimmunization, and prevent transplant rejection and platelet refractoriness in a subject. The disclosure is partly based on the surprising discovery that a transient low-dose methotrexate (TLD-MTX) regimen can induce long-term platelet antigen-specific immune tolerance in a subject. The TLD-MTX regimen comprises administering a first methotrexate (MTX) treatment concurrently with an initial platelet transfusion, followed by two consecutive daily doses of MTX. Inventors have further optimized the dose and timing of administration of TLD-MTX regimen and platelet transfusion. The disclosed TLD-MTX regimen induces long-term platelet antigen-specific immune tolerance, particularly to major histocompatibility complex (MHC) antigens, to prevent or reduce alloimmunization, without side effects of general immunosuppression. The disclosure further provides methods to prevent or reduce alloimmunization and alloimmune sequelae, including platelet refractoriness and subsequent transplant rejection.

The methods and compositions disclosed herein induce tolerance to allogeneic platelets which significantly benefit transfusion or transplant recipients by reducing the risk of alloimmunization and prevent subsequent transplant rejection and platelet refractoriness. This would result in more effective transfusions, decreased patient exposure, conservation of the blood supply, and lower healthcare costs. The methods and composition provided herein mitigate alloimmunization and alloimmune sequelae, particularly in high-risk populations, and further may be applicable to other autoimmune disorders.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### Terminology

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase ‘consisting essentially of’ limits the scope of a claim to the recited components in a composition or the recited steps in a method as well as those that do not materially affect the basic and novel characteristic or characteristics of the claimed composition or claimed method. The phrase ‘consisting of’ excludes any component, step, or element that is not recited in the claim. The phrase ‘comprising’ is synonymous with ‘including’, ‘containing’, or ‘characterized by’, and is inclusive or open-ended. ‘Comprising’ does not exclude additional, unrecited components or steps.

As used herein, when referring to any numerical value, the term ‘about’ means a value falling within a range that is ±10% of the stated value.

Ranges can be expressed herein as from ‘about’ one particular value, and/or to ‘about’ another particular value. When such a range is expressed, a further aspect includes from the one particular value and/or to the other particular value. Similarly, when values are expressed as approximations, by use of the antecedent ‘about,’ it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as ‘about’ that particular value in addition to the value itself. For example, if the value ‘10’ is disclosed, then ‘about 10’ is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms ‘optional’ or ‘optionally’ means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

The present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein “alloimmunization” refers to an immune response to foreign antigens or alloantigens after exposure to genetically different cells or tissues, such as during, for e.g., blood transfusion or transplantation. Alloimmunization may result in the rejection of transfused or transplanted tissues, such as platelets, which leads to platelet refractoriness. Alloantigens include but are not limited to blood group substances (A, B, or AB) on erythrocytes and histocompatibility antigens expressed on white blood cells and platelets, and other blood-cell associated minor antigens.

Human Leukocyte Antigen (HLA) markers are found on the membranes of many different cell types, including white blood cells. HLA is the major histocompatibility complex (MHC) in humans and contributes to the recognition of self v. non-self. Recognition by a transfusion recipient's immune system of differences in HLA antigens on the surface of transfused cells may be the first step in the rejection of subsequently transfused or transplanted tissues. Therefore, the phenomena of alloimmunization of recipients against HLA markers on donor blood is a major problem in transfusion medicine today. This issue commonly arises in recipients of blood products due to the generation of antibodies against white blood cell HLA antigens in donor blood but generation of antibodies to minor antigens could also contribute.

Platelets also express on their surface low levels of these HLA antigens. When a recipient of a whole blood or a blood component that contains donor white blood cells is transfused, the recipient may produce antibodies against the HLA antigens on the transfused donor's white blood cells. These antibodies may also lead to recognition and clearance of subsequently transfused platelets that carry this same marker. When this occurs, it becomes necessary to HLA match the platelet donor and recipient to assure that the recipient receiving the transfusion is able to maintain an adequate number of donor platelets in circulation. Finding an HLA-compatible donor is often a complicated, expensive and difficult procedure because of the complexity of the HLA system. Large numbers of potential platelet donors must be HLA-typed in order to have an available platelet donor registry that will contain compatible donors for most patients. In cases where recipients are very heavily transfused with blood or blood products from multiple donors and antibodies to many different HLA markers are generated, or where no suitable HLA-compatible platelet donor is available, death due to bleeding may occur.

As used herein “alloimmune sequelae” refers to any complications or condition that results from an alloimmunization during, for e.g., blood transfusion or transplantation. Such complications or conditions can be chronic and or acute.

As used herein, the term “allogeneic transfusion” refers to transfusion involving collecting blood components from a donor and then infusing the collected blood components into a recipient who is different from the donor. Sometimes, however, the recipient of an allogeneic transfusion experiences a disease commonly known as graft versus host disease (GVHD). In graft versus host disease, T cells, which may accompany the blood components, are infused into the recipient and “recognize” that the recipient's body is “foreign” from that of the donor's. These T cells “attack” healthy cells in the recipient's body, rather than performing a normal immunological protective function.

As used herein, the term “leukoreduction” or “leukoreduced” refer to removal of white blood cells (WBCs) from cellular components to reduce the risk of HLA alloimmunization, Cytomegalovirus (CMV) transmission, and febrile nonhemolytic transfusion reactions. Leukoreduction can be performed by filtration prior to component storage (pre-storage leukoreduction) or during the transfusion (bedside filtration). For apheresis-derived platelets, leukoreduction is often performed by cell separation during the apheresis collection. For whole blood, whole blood-derived platelets, and red blood cells (RBCs), leukoreduction is performed using third-generation leukoreduction filters, which are commercially available as an integral part of a whole blood collection kit or as a separate, sterilely docked device.

As used herein “platelet refractoriness” or “platelet transfusion refractoriness” refer to the repeated failure to achieve the desired level of blood platelets in a patient following a platelet transfusion. One of the causes for platelet refractoriness can be alloimmunization. Platelet refractoriness occurs when a recipient fails to obtain a satisfactory response to two or more successive platelet transfusions. In clinical practice, there is usually little doubt when patients are failing to have satisfactory responses to a platelet transfusion, as indicated by no increase in platelet count on the day of or the day after a platelet transfusion.

As used herein, an “antigen” can be a molecule, or a moiety, that can bind to a specific antibody. In some aspects, antigen can comprise an immunogen part of a donor cell.

As used herein, the term “hematopoietic stem cells” or “HSCs” generally refers to immature hematopoietic cells having the capacity to self-renew and to differentiate into more mature blood cells comprising but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), other myeloid cells (e.g., monocytes, macrophages, dendritic cells), and lymphocytes (B cells, T cells and natural killer cells). HSCs are interchangeably described as stem cells throughout the specification. Such cells may include CD34⁺ cells. CD34⁺ cells are immature cells that express the CD34 cell surface marker. CD34⁺ cells are believed to include a subpopulation of cells with the stem cell properties defined above. Such cells may include CD150⁺ cells. CD150⁺ cells are immature cells that express the CD150 cell surface marker. The CD150⁺ cells may have long-term, multilineage-reconstituting ability. HSCs may also include pluripotent stem cells, multipotent stem cells (e.g., a lymphoid stem cell), and/or stem cells committed to specific hematopoietic lineages. The stem cells committed to specific hematopoietic lineages may be of T cell lineage, B cell lineage, dendritic cell lineage, Langerhans cell lineage and/or myeloid tissue-specific macrophage cell lineage. In addition, HSCs may also include long term HSC (LT-HSC) and short term HSC (ST-HSC). ST-HSCs are more active and more proliferative than LT-HSCs. However, LT-HSC have unlimited self-renewal ability (i.e., they survive throughout adulthood), whereas ST-HSC have limited self-renewal ability (i.e., they survive for only a limited period of time). Any of these HSCs can be used in any of the methods described herein. Hematopoietic stem cells may be obtained from blood products. A blood product includes a product obtained from the body or an organ of the body containing cells of hematopoietic origin. Such sources include un-fractionated bone marrow, umbilical cord, peripheral blood, liver, thymus, lymph and spleen. All of the aforementioned crude or un-fractionated blood products can be enriched for cells having hematopoietic stem cell characteristics.

As used herein, “individual”, “subject”, “host”, and “patient” can be used interchangeably herein and refer to any mammalian subject for whom diagnosis, treatment, prophylaxis, or therapy is desired, for example, humans, pets, livestock, horses, or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human. In other aspects, the subject can be a subject planning to undergo transfusion, organ transplant, or stem cell transplant.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to prevent, mitigate, cure, heal, alleviate, relieve, alter, remedy, ameliorate, improve, or affect the disorder, the symptom of the disease, or the predisposition toward the disease or disorder. Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

As used herein a first allogeneic transfusion may be an initial allogeneic transfusion concurrently given with first dose of TLD-MTX. In some aspects, the initial transfusion may be the first transfusion the subject has ever received. In another aspect, the subject may have received one or more prior transfusions before the allogeneic transfusion concurrently given with first dose of TLD-MTX.

Unless otherwise defined, all technical terms used herein have the same meaning as commonly understood by one of ordinary skill in the art to which this disclosure belongs.

### Methods

The present disclosure encompasses a method of preventing or reducing immune response in a subject. The method generally comprises administering an initial low dose of MTX concurrently with a first platelet transfusion. The subject may be planning to undergo or have undergone a transplant or a transfusion. The methods described herein can be used to prevent or reduce complications associated with a transfusion, for e.g., platelet transfusion, or a transplantation, for e.g., HSCT. A subject in need of a transfusion or transplantation can also include subjects undergoing a therapeutic treatment, such as chemotherapy or radiation therapy. One example of such complication is alloimmunization to platelet transfusions which can lead to serious complications, including future transplant rejection and platelet refractoriness to subsequent transfusions. In one example the subject may be at high risk for platelet alloimmunization. Current clinical practices, such as matching donor and recipient are hindered by challenges including the short shelf-life of platelets and the high costs of testing. Despite modern leukoreduction techniques, the risk of alloimmunization remains, especially for recipients of multiple transfusions. The treatment regimens with TLD-MTX described herein is a safe and effective strategy to induce long-term, immune tolerance to prevent platelet alloimmunization and protect against future platelet refractoriness and transplants.

In one aspect of the disclosure, the method comprises preventing or reducing alloimmunization and/or alloimmune sequelae. The method comprises administering in a subject in need thereof, a first dose of MTX concurrently with a first allogeneic transfusion. The method may further comprise administering a second dose of MTX, and/or a third dose of MTX.

In some aspects, the method of preventing or reducing alloimmunization and/or alloimmune sequelae comprises, administering in a subject in need thereof a first dose of MTX concurrently with a first allogeneic platelet transfusion, a second dose of MTX, and a third dose of MTX. In such aspects, the first allogeneic platelet transfusion comprises transfusion of leukoreduced or non-leukoreduced platelets.

In another aspect, the method of the present disclosure comprises inducing immune tolerance in a subject in need of allogeneic transfusion. The method comprises administering a first dose of MTX concurrently with the allogeneic transfusion and administering a second dose of MTX. The method may further comprise administering a third dose of MTX to the subject.

The method disclosed herein further comprises increasing the efficacy of multiple allogeneic transfusions. In such aspects, the method comprises administering in a subject in need thereof, a first dose of MTX concurrently with the first allogeneic transfusion, and administering a second dose of MTX, thereby inducing immune tolerance in the subject to subsequent allogeneic transfusion. The method may further comprise administering a third dose of MTX to the subject.

Further provided is a method of reducing risk of bone marrow transplant rejection in a subject. The method comprises administering a first dose of MTX concurrently with platelet transfusion in a subject planning to undergo bone marrow transplant. In such aspects, administering MTX concurrently with platelet transfusion may reduce the risk of bone marrow transplant rejection. The method may further comprise administering a second dose of MTX, and/or a third dose of MTX.

In yet another aspect, the method disclosed herein prevents or reduces stem cell rejection in a subject. The method comprises administering in a subject planning to undergo or have undergone a stem cell transplant, for e.g., hematopoietic stem cell transplant (HSCT), a first dose of MTX concurrently with the first allogeneic transfusion, and administering a second dose of MTX, thereby inducing immune tolerance in the subject to subsequent allogeneic transfusion. The method may further comprise administering a second dose of MTX and/or a third dose of MTX to the subject.

In a further aspect, the method disclosed herein prevents or reduces GVHD in a subject. The method comprises administering in a subject planning to undergo or have undergone a transfusion or a transplantation, for e.g., hematopoietic stem cell transplant (HSCT), a first dose of MTX concurrently with the first allogeneic transfusion, and administering a second dose of MTX, thereby inducing immune tolerance in the subject to subsequent allogeneic transfusion. The method may further comprise administering a third dose of MTX to the subject. The first allogenic transfusion comprises allogeneic platelet transfusion.

A method of reducing risk of bone marrow transplant rejection is further provided. The method comprises administering to a subject planning to undergo or have undergone bone marrow transplant, a first dose of MTX concurrently with a first allogeneic transfusion. The first allogeneic transfusion may comprise a first allogeneic platelet transfusion. The method may further comprise administering a second dose of MTX and/or a third dose of MTX to the subject.

The first allogeneic transfusion described herein may comprise transfusion with allogeneic platelets. However, allogeneic blood or allogeneic hematopoietic stem cells (HSC) are also contemplated for use in first allogeneic transfusion. The first allogeneic transfusion may be an initial allogeneic transfusion concurrently given with first dose of MTX. In some aspects, the initial transfusion may be the first transfusion the subject has ever received. In another aspect, the subject may have received one or more prior transfusions before the allogeneic transfusion concurrently given with first dose of MTX. The dose of the MTX may further be TLD-MTX.

The methods disclosed herein may further comprise administering at least a second allogeneic transfusion. The methods may comprise multiple subsequent allogeneic transfusions, for e.g., second, third, fourth, fifth, sixth, seventh, eighth, ninth, tenth, or more transfusions in the subject. In certain aspects, subsequent transfusion comprises allogeneic platelet transfusions.

A subject according to the present application, may be an individual with or at risk for depleted or limited blood cell levels. An event that changes blood cell levels in a subject includes, for example, anemia, trauma, chemotherapy, bone marrow transplant, surgery, congenital, radiation therapy and/or radiation in the environment. For example, the subject may have anemia or blood loss due to, for example, trauma. Subject may also be a bone marrow donor prior to bone marrow harvesting or a bone marrow donor after bone marrow harvesting, or a recipient of a bone marrow transplant. In another aspect, subjects may have thrombocytopenia, such as immune thrombocytopenia (ITP). In another aspect, the subject may be planning to undergo or have undergone an HSC transplantation. Subjects, for example may be diagnosed or be at risk for hematologic malignancy. Hematologic malignancy may correspond to one or more of acute lymphocytic leukemia, acute myelogenous leukemia, chronic myelogenous leukemia, multiple myeloma, lymphoma, Hodgkin's lymphoma, non-Hodgkin lymphoma, myelodysplastic syndrome, myelofibrosis, and blastic plasmacytoid dendritic cell neoplasm (BPDCN).

In some aspects, the subject is at least 3, at least 4, at least 5, at least 6, at least 7, at least 8, at least 9, at least 10, at least 11, or at least 12 months of age. In one aspect, the subject is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 years of age. In some aspects, the subject is approximately 18 years of age or older. In another aspect, the subject is approximately 16 years of age or older. In other aspects, a subject is approximately 13 years of age or older. In some aspects, the subject is approximately 6 years of age or older. In some aspects, the subject is approximately 3 years of age or older. In some aspects, the subject is approximately 1 year of age or older. In another aspect, the subject is approximately 9 months of age or older. In some aspects, the subject is approximately 6 months of age or older. In some aspects, a subject is approximately 3 months of age or older. In another aspect, the subject is at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, or at most 80 years of age. In some aspects, the subject is at most 65 years of age. In some aspects, the subject is at most 70 years of age. In another aspect, the subject is at most 75 years of age. In some aspects, the subject is from between approximately 3 months to approximately 18 years of age. In certain aspects, the subject is from approximately 18 years to approximately 65 years of age. In some aspects, a subject is from approximately 18 years to approximately 75 years of age. In some aspects, a subject is from approximately 66 years to approximately 75 years of age, or more.

Platelets for transfusion may be obtained using any known methods in the art. Whole blood collected from donors for transfusion into recipients can be separated into components including red blood cells, white blood cells, platelets, and plasma, using apheresis, centrifugation procedures, or other methods. Platelets thus obtained may be immediately used or stored for later use. In one aspect, the platelets used in the methods described herein, may be leukoreduced platelets or non-leukoreduced platelets. Leukoreduction of the platelets can be performed using any known methods in the art. Non-leukoreduced platelets may comprise white blood cells.

In one aspect, platelets may be obtained from an allogeneic donor, referred herein as allogeneic platelets. Allogenic donor, for example may have at least one HLA mismatch to the human subject. HLA mismatch may be at an allele selected from: HLA-A, HLA-B, HLA-C, DRB-1, and any combination thereof. In some aspects, the allogeneic donor that has at least one HLA mismatch is 6/8 HLA-mismatched relative to the subject or is 7/8 HLA-mismatched relative to the subject. In some aspects, the allogeneic donor is 7/8 HLA-mismatched relative to the subject. In some aspects, the allogeneic donor that has at least one HLA mismatch relative to the human subject has a mismatched HLA allele as a result of the allogeneic donor being homozygous for the HLA allele while the human subject is heterogeneous for the HLA allele. In some aspects, the allogeneic donor that has at least one HLA mismatch relative to the human subject has a mismatched HLA allele as a result of the allogeneic donor being heterozygous for the HLA allele while the human subject is homozygous for the HLA allele. In some aspects, the allogeneic donor that has at least one HLA mismatch relative to the human subject has a mismatched HLA allele as a result of both the allogeneic donor and the human subject being heterozygous for the HLA allele.

The first, second and third MTX doses described herein may be administered as a daily dose to the subject. The MTX may be a TLD-MTX dose. The dose of TLD-MTX may be about 0.1 mg/kg to about 7 mg/kg.

The methods of the present disclosure may further comprise administering additional MTX doses, optionally TLD-MTX. The subject may be administered additional MTX doses on 4-20 days after administration of the first dose. In one aspect, the methods comprise administering a MTX dose on 4^{th}, 5^{th}, 6^{th}, 7^{th}, 8^{th}, 9^{th}, 10^{th} , 11^{th}, 12^{th}, 13^{th}, 14^{th}, 15^{th}, 16^{th}, 17^{th}, 18^{th}, 19^{th}, and/or or 20^{th} day after administration of the first dose. In some aspects, the methods comprise administering a daily dose of MTX on 4^{th}, 5^{th}, 6^{th} , 7^{th}, 8^{th}, 9^{th}, 10^{th}, and/or 11^{th} day after administration of the first dose. Doses may be administered on consecutive days or on non-consecutive days.

MTX, optionally TLD-MTX, in one aspect, is administered as 1-12 consecutive daily doses. In one example, MTX may be administered as 3 consecutive daily doses. The first dose of MTX may be given concurrently, or immediately before or after the first platelet transfusion. For example, the first MTX dose may be given within 1-60 minutes of the first platelet transfusion. The first MTX dose may be given within about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes of the first platelet transfusion. In one specific aspect, the first MTX dose may be given within about 15 minutes before or after the first platelet transfusion. The following doses may be administered on consecutive days.

MTX, optionally TLD-MTX, in one aspect, is administered as 1-12 non-consecutive doses. In one example, MTX may be administered as non-consecutive daily doses. The first dose of MTX may be given concurrently, or immediately before or after the first platelet transfusion. For example, the first MTX dose may be given within 1-60 minutes of the first platelet transfusion. The first MTX dose may be given within about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes of the first platelet transfusion. In one specific aspect, the first MTX dose may be given within about 15 minutes before or after first platelet transfusion. The following doses may be administered on non-consecutive days.

In certain aspects, the first MTX may be administered before the first platelet transfusion. MTX may be administered about 10 minutes to about 120 minutes, about 3 hours to about 24 hours, about 1 day to about 5 days before administration of the first platelet transfusion. The first MTX dose may be given about 10 minutes, about 15 minutes, about 30 minutes, about 45 minutes, about 60 minutes, about 1.5 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, or about 4 days or about 5 days before the first platelet transfusion.

Conventional methods, known to those of ordinary skill in the art of medicine, can be used to administer the MTX disclosed herein to a subject. In one example, the MTX is a TLD-MTX. In some aspects, MTX herein can be administered to a subject, for e.g., as a bolus or by continuous infusion over a period of time, by intraperitoneal, intramuscular, intracerebrospinal, subcutaneous, intravenous, intra-arterial, intra-articular, intrasynovial, intrathecal, oral, inhalation, topical routes, or other mode of administration. In some aspects, MTX herein may be administered to a subject intraperitoneally.

A suitable, non-limiting example of a dosage according to the present disclosure may be from about 0.1 mg/kg to about 10 mg/kg, more particularly about 0.1 mg/kg to about 7 mg/kg. In general, however, doses employed for human treatment typically may be in the range of about 0.0001 mg/kg/day to about 0.0010 mg/kg/day, about 0.0010 mg/kg/day to about 0.010 mg/kg/day, about 0.010 mg/kg/day to about 0.10 mg/kg/day, about 0.10 mg/kg/day to about 1.0 mg/kg/day, about 1.00 mg/kg/day to about 3 mg/kg/day, about 1 mg/kg/day to about 10 mg/kg/day. For example, the dosage may be about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In one aspect, the MTX dose administered to the subject may be about 5 mg/kg.

Dosage amount and interval may be adjusted individually to provide plasma levels of the MTX, which are sufficient to maintain therapeutic or prophylactic effect. For example, the MTX thereof may be administered once per week, several times per week (e.g., every other day), once per day or multiple times per day, depending upon, among other things, the mode of administration, the specific indication being treated and the judgment of the prescribing physician. Skilled artisans will be able to optimize effective dosages without undue experimentation.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts. For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

The methods described herein, enhance immunotolerance in the subject. The immunotolerance induced in the subject may actively suppress alloimmunity during subsequent transfusions or transplants. In one aspect the methods of the present disclosure prevent or reduce alloimmunization in the subject for at least about 1-52 weeks. In some aspects, the methods prevent or reduce alloimmunization in the subject for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, or more. Immunotolerance in a subject can measured using any known method in the art.

In one aspect, the methods of present disclosure, result in enhancement of level of one or one or more of regulatory B cells, and regulatory T cells in the subject. In some aspects, the method of administration of MTX, for example TLD-MTX, described herein results in increase in level of one or one or more of regulatory B cells, and regulatory T cells at least about 10%, e.g., about 20%, about 30%, about 40%, about 50%, about 60%, about 70%, about 80%, about 90%, about 95% or above, as compared to the level before administration of the MTX or a control subject not receiving the MTX. In some aspects, the methods described herein results in increase in the level of one or one or more of regulatory B cells and regulatory T cells, by at least about 10%. Any known method in the art can be used to measure the levels of regulatory B cells and regulatory T cells in the subject, a non-limiting example of which include fluorophore detection using class I and class II MHC tetramers. Regulatory T cells may include FoxP3/CD25 expressing T Regulatory cells, follicular helper T Regulatory cells, and/or CTLA-4 and TGF-beta expressing T Regulatory cells.

In another aspect, the methods of the present disclosure, result in reduction of T cell levels, T cell activation, and/or T follicular helper cells (T_{F}H) levels. In some aspects, the method of administration of MTX, for example TLD-MTX described herein results in decreased T cell levels, T cell activation, and/or T follicular helper cells levels at least about 10%, e.g., about 20%, about 30%, about 40%, about 50%, about 60%, about 70%, about 80%, about 90%, about 95% or above, as compared to the level before administration of the MTX or a control subject not receiving the MTX. In some aspects, the methods described herein results in decrease in the level of one or one or more of T cell levels, T cells activation, and/or T follicular helper cells (T_{F}H) levels, by at least about 10%.

In some aspects, the methods of present disclosure, result in enhancement of levels of one or one or more of IL-10, and TGF-beta in the subject. In some aspects, the method of administration of MTX, for example TLD-MTX described herein results in increase in level of one or one or more of IL-10, and TGF-beta at least about 10%, e.g., about 20%, about 30%, about 40%, about 50%, 60%, 70%, 80%, 90%, 95% or above, as compared to the level before administration of the MTX or a control subject not receiving the MTX. In some aspects, the methods described herein results in increase in the level of one or one or more of IL-10, and TGF-beta by at least about 10%. Any known method in the art can be used to measure the levels of IL-10, and TGF-beta in the subject, a non-limiting example of which include flow cytometry, immunohistochemistry (IHC), and bead-based multiplex assays.

In some aspects, the methods of present disclosure, result in reduction of level of one or one or more of TNF-alpha, IFN-gamma, IL-2, IL-12, IL-4, IL-5, and IL-13 in the subject. In some aspects, the method of administration of TLD-MTX described herein results in decrease in level of one or one or more of TNF-alpha, IFN-gamma, IL-2, IL-12, IL-4, IL-5, and IL-13 by at least about 10%, e.g., about 20%, about 30%, about 40%, about 50%, about 60%, about 70%, about 80%, about 90%, about 95% or above, as compared to the level before administration of the MTX (for example, TLD-MTX) or a control subject not receiving the MTX. In some aspects, the methods described herein results in decrease in the level of one or one or more of TNF-alpha, IFN-gamma, IL-2, IL-12, IL-4, IL-5, and IL-13 by at least about 10%. Any known method in the art can be used to measure the levels of TNF-alpha, IFN-gamma, IL-2, IL-12, IL-4, IL-5, and IL-13 in the subject, a non-limiting example of which include flow cytometry, IHC, and bead-based multiplex assays.

In some aspects, the methods of present disclosure, result in reduction of total alloantibody level in the subject. In some aspects, the method of administration of MTX, optionally TLD-MTX, described herein results in decrease in total alloantibody level by at least about 10%, e.g., about 20%, about 30%, about 40%, about 50%, about 60%, about 70%, about 80%, about 90%, about 95% or above, as compared to the level before administration of the MTX or a control subject not receiving the MTX. In some aspects, the methods described herein results in decrease in the total alloantibody level by at least about 10%. Any known method in the art can be used to measure the levels of total alloantibody level in the subject, a non-limiting example of which include flow cytometry, enzyme-linked immunosorbent assay, fluorophore, or enzymatic detection with or without using class I and class II MHC tetramers.

In another aspect, the methods of present disclosure, result in skewing the alloantibody response in the subject towards antibody isotypes that are less inflammatory or more immunotolerant. For example, in mice, administering MTX, optionally TLD-MTX concurrently with the first platelet transfusion can reduce the level of one or more of IgK, IgG3, IgG2b, and IgG2a/c and/or enhance the level of IgG1. In alternate example, in human, administering MTX, optionally TLD-MTX concurrently with the first platelet transfusion can alter the level of one or more of IgG1, IgG2, IgG3, and/or IgG4. The reduction or enhancement of these isotypes be at least about 10%, e.g., about 20%, about 30%, about 40%, about 50%, about 60%, about 70%, about 80%, about 90%, about 95% or above, as compared to the level before administration of the MTX (for example, TLD-MTX) or a control subject not receiving the MTX.

The methods of the present application may be used as a supplemental treatment or therapy to chemotherapy and/or radiation therapy. For example, the subject may be a subject that has been subjected to, is undergoing or is expected to undergo an immune cell depleting treatment such as chemotherapy, radiation therapy or serving as a donor for a bone marrow transplant. Bone marrow is one of the most prolific tissues in the body and is therefore often the organ that is initially damaged by chemotherapy drugs and radiation. The result is that blood cell production is rapidly destroyed during chemotherapy or radiation treatment, and chemotherapy or radiation must be terminated to allow the hematopoietic system to replenish the blood cell supplies before a patient is re-treated with chemotherapy. Therefore, the methods of the present application may be applied to such subjects. In some cases, the subject has received bone marrow transplantation.

In certain aspects, the methods described herein may be combined with other treatments. For example, after the first, second, or third dose of MTX, optionally TLD-MTX, a therapeutic agent may be administered to the subject. Therapeutic agents may comprise a GVHD prophylactic agent such as tacrolimus, or sirolimus, antibiotics, anti-pyrectics, antimicrobials, antifungals, NSAIDs, chemotherapeutic and/or anticancer agents.

In a specific aspect, the methods of treatment disclosed herein exclude administration of human acid alpha-glucosidase. The methods of treatment may further exclude administration of alemtuzumab, rituximab, or an anti-thymocyte globulin polyclonal antibody. In one aspect, the method of treatment of the present disclosure excludes heart allografting.

Any of the methods described herein can further comprise adjusting the treatment performed to the subject based on the results obtained from the methods disclosed herein. Adjusting treatment includes, but are not limited to, changing the dose and/or administration of the MTX, for example TLD-MTX used in the current treatment, or applying a new therapy to the subject, which can be either in combination with the current therapy or replacing the current therapy.

### Compositions

Some aspects of the disclosure further encompass a composition comprising MTX, for example, TLD-MTX. The compositions of the present disclosure may be formulated for one or more routes of administration. Suitable routes of administration may, for example, include oral, rectal, transmucosal, transnasal, intestinal, and/or parenteral delivery. In some aspects, compositions herein formulated can be formulated for parenteral delivery. In some aspects, compositions herein formulated can be formulated for intraperitoneal, intramuscular, subcutaneous, intramedullary, intravenous, and/or intranasal injections.

In certain aspects, compositions of the present disclosure may be a pharmaceutical composition. In certain aspects, the pharmaceutical compositions can include pharmaceutically acceptable carriers, excipients, and/or stabilizers in the form of lyophilized formulations or aqueous solutions. In some aspects, acceptable carriers, excipients, and/or stabilizers are nontoxic to recipients at the dosages and concentrations used, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN, PLURONICS or polyethylene glycol (PEG).

In one instance, the composition may comprise MTX at a dosage of 0.1 ng/kg to about 5000 mg/kg. In general, however, doses employed for example for an adult human treatment, typically may be in the range of 0.0001 mg/kg/day to 0.0010 mg/kg/day, 0.0010 mg/kg/day to 0.010 mg/kg/day, 0.010 mg/kg/day to 0.10 mg/kg/day, 0.10 mg/kg/day to 1 0.0 mg/kg/day, 1 0.00 mg/kg/day to about 200 mg/kg/day, 200 mg/kg/day to about 5000 mg/kg/day. For example, the dosage may be about 1 mg/kg/day to about 100 mg/kg/day, such as, e.g., 2-10 mg/kg/day, 10-50 mg/kg/day, or 50-100 mg/kg/day. The dosage can also be selected from about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1 100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, 2300 mg/kg, 2400 mg/kg, 2500 mg/kg, 2600 mg/kg, 2700 mg/kg, 2800 mg/kg, 2900 mg/kg, 3000 mg/kg, 3500 mg/kg, 4000 mg/kg, or 5000 mg/kg.

In certain aspects, pharmaceutical compositions for use in accordance with the present disclosure may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof. Formulations for injection herein further may be presented in unit dosage form, e.g., in ampoules or in multidose containers with optionally, an added preservative. In some aspects, compositions herein may be suspensions, solutions, or emulsions in oily or aqueous vehicles, and/or may contain formulatory agents such as suspending, stabilizing and/or dispersing agents.

The compositions disclosed herein may be manufactured by processes well known in the art, e.g., by means of conventional mixing, dissolving, granulating, dragee-making, levigating, emulsifying, encapsulating, entrapping or lyophilizing processes.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are proteinrally understood in the art to be optimal.

### Kits

Further provided herein is a kit. The kit may comprise a composition comprising MTX, for example TLD-MTX, and instructions for administering the composition to a subject in need thereof. The kit could further comprise allogeneic platelets for transfusion. In some aspects, the kits can further comprise a sterile, pharmaceutically acceptable carrier, buffer, or other diluent. The kits provided herein generally include instructions for carrying out the methods. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The disclosed kit may have a single container that contains the MTX, for example TLD-MTX with or without any additional components, or they may have distinct containers for each desired agent. Where combined therapeutics are provided, a single solution may be pre-mixed, either in a molar equivalent combination, or with one component in excess of the other. Alternatively, each of the MTX and other components of the kit, for example platelets may be maintained separately within distinct containers prior to administration to a patient.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

The containers of the kit will generally include at least one vial, test tube, flask, bottle, syringe or other container means, into which the disclosed MTX, optionally TLD-MTX, and any other desired agent, may be placed and, preferably, suitably aliquoted. Where separate components are included, the kit will also generally contain a second vial or other container into which these are placed, enabling the administration of separated designed doses. The kits may also comprise a second/third container means for containing a sterile, pharmaceutically acceptable buffer or other diluent.

In certain aspects, the kit may also contain a means by which to administer the MTX, optionally TLD-MTX. For example, one or more needles or syringes, or even an eye dropper, pipette, or other such like apparatus, from which the formulation may be injected into the animal or applied to a diseased area of the body. The kits of the present disclosure will also typically include a means for containing the vials, or such like, and other component, in close confinement for commercial sale, such as, e.g., injection or blow-molded plastic containers into which the desired vials and other apparatus are placed and retained.

Having described several aspects, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

### EXAMPLES

The Examples that follow are illustrative of specific aspects of the invention, and various uses thereof. They set forth for explanatory purposes only and are not to be taken as limiting the invention.

Methotrexate (MTX) has been used in a combination therapy to induce long-term immune tolerance to an enzyme replacement therapy for a rare lysosomal storage disease. The induction of immune tolerance by MTX is not obvious in every scenario as it does not work with every antigen. Its demonstrated use to induce immune tolerance is limited to a few antigens, and there is a general skepticism whether it can induce immune tolerance to every antigen or to other fields of medicine. Anecdotal evidence has shown that it is not effective in inducing immune tolerance to viral antigens. The immunogenicity of the antigen may play an important role on whether immune tolerance can be achieved.

The examples described herein provide methods for induction of long-term immune tolerance to prevent platelet alloimmunization, aiming to mitigate alloimmune sequelae such as subsequent transplant rejection and platelet refractoriness. The experimental approach uses MHC tetramers, which allows for the examination of alloantigen-specific T cells, and tracking the behavior of alloreactive B cells to tease out the mechanism by which TLD-MTX induces tolerance. Further described are assessments on the durability of immune tolerance induction (ITI) by TLD-MTX through multiple allogeneic platelet transfusions and the efficacy of this tolerance induction in the presence of pre-existing alloantibodies. Evaluation on functional outcomes, including its protective effects on subsequent transplants and platelet transfusions and on graft-versus-host disease (GVHD) is further provided.

### Example 1: Mouse Model

A robust mouse model of platelet transfusion and alloimmunization was established, along with a comprehensive set of assays to evaluate humoral and cellular alloimmune responses, particularly against the primary platelet antigen, MHC. The examination of alloreactive B cells is challenging due to their low frequency and responses being masked by the general B cell population. However, an experimental strategy was optimized and demonstrated to track these rare, allogeneic donor-specific B cells (allo-B cells) using MHC tetramers, and which was able to differentiate responses between class I and class II MHC antigens. This approach allowed to reproducibly identify a discrete population of allo-B cells and investigate changes in their activation, antigen experience, germinal center (GC) formation, and memory development in response to different allogeneic platelet transfusion conditions. These assays can easily be adapted to evaluate the role of regulatory B cells (B_{Regs}) in immunotolerant environments or during active induction of immune tolerance. Total T cell responses and various antigen presenting cells were previously measured in response to platelet transfusion and the induction of partial tolerance. This includes examining T cell activation, cytokine production, and CD4⁺ FoxP3⁺ regulatory T cells (T_{Regs}). While T cell responses to transfusion are sufficiently robust for detection, examining T_{Regs}is challenging due to their rarity. However, the behavior of rare alloantigen-specific T_{Regs} using class II MHC tetramers can be tracked. A highly sensitive alloantibody detection screen that differentiates responses between class I and class II MHC was further developed. This screen measures total alloantibodies and their individual isotypes, allowing detection of shifts in antibody effector function that may be skewed towards tolerance. Cellular architecture and localization of immune cells will be examined through immunohistochemistry. Cytokine type and levels will be assessed using multiplexing methods, enabling evaluation of subtle changes in an inflammatory and immunotolerant cytokine milieu. Finally, the potency of antibodies by functional outcomes in models of platelet refractoriness, GVHD, and bone marrow transplant will be measured.

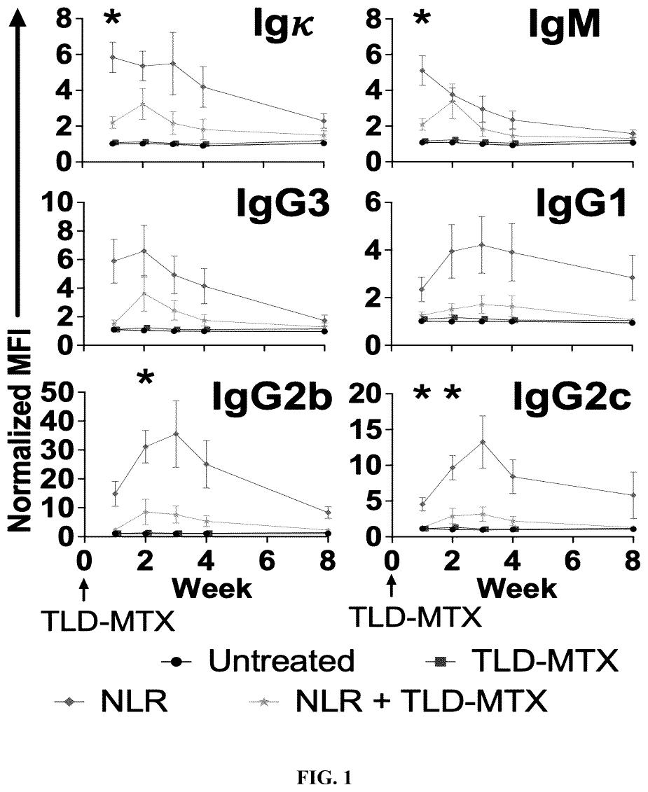

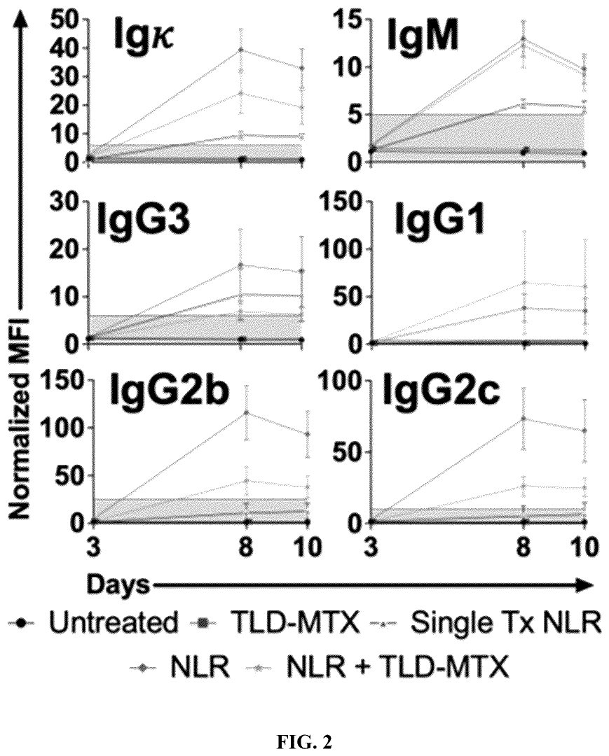

It was demonstrated that TLD-MTX, when administered concurrently with the initial platelet transfusion, diminished total (Igc) and isotype alloantibody responses, and these suppressed responses were durable over time (FIG. 1). Of the isotypes, the ones with the greatest inflammatory function (IgG2b and IgG2c) were significantly suppressed. Upon a recall response with a secondary platelet transfusion 12 weeks later, total alloantibody responses clearly showed a reduced trend (FIG. 2). This was associated with a reduced trend in inflammatory isotypes IgG3, IgG2b, and IgG2c, and a skewed isotype profile towards regulatory IgG1. This indicates that TLD-MTX successfully induced lasting immune tolerance to allogeneic platelet transfusions. These examples aim to evaluate the cellular mechanisms of tolerance by TLD-MTX, focusing on alloreactive B cells, T cells, and the induction of regulatory humoral and cellular immunity. The efficacy and durability of TLD-MTX immune tolerance induction (ITI) will be investigated by examining alloantibody responses across multiple transfusions and in the context of pre-existing alloantibodies. Finally, functional outcomes will be assessed to better understand the utility of TLD-MTX ITI in preventing platelet alloimmunization sequelae.

### Example 2: Determination of Immune-Mediated Mechanism Driving TLD-MTX Tolerance Induction

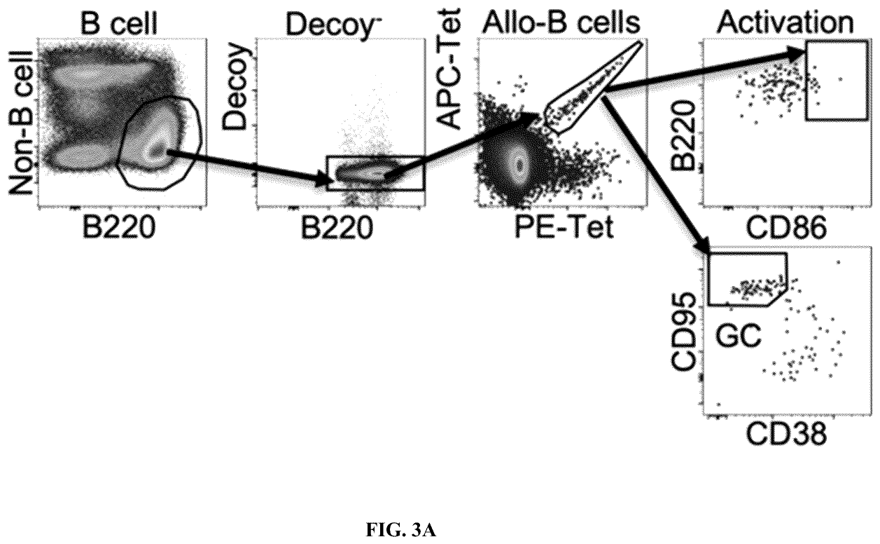

It was hypothesized that TLD-MTX ITI will diminish responses to donor-specific alloreactive immunity and induce regulatory B cells and T cells in acute responses as well as after multiple transfusions. FIG. 1 shows that when TLD-MTX ITI was administered alongside an allogeneic non-leukoreduced platelet transfusion (NLR+TLD-MTX), total and isotype alloantibodies were diminished over time relative to mice that received non-leukoreduced platelet transfusion alone (NLR). Moreover, TLD-MTX appeared to have established durable immune tolerance, as there was a reduced trend when recipients received only a secondary platelet transfusion 12 weeks later (NLR+TLD-MTX, FIG. 2). While TLD-MTX broadly reduced all isotypes after a primary platelet transfusion, the responses clearly showed a trend of skewing towards the regulatory IgG1 isotype after the secondary challenge. To better understand how TLD-MTX is suppressing these alloantibody responses, alloreactive B cells and T cells will be examined. The prior work with MHC tetramers has demonstrated that a single allogeneic platelet transfusion elicits robust expansion of allo-B cells and upregulates allo-B cell activation and GC formation (FIG. 3A-3D). TLD-MTX may be inhibiting these responses which subsequently diminishes alloantibody expression. GCs are critical sites for somatic hypermutation and class switching which promotes B cell maturity and affinity maturation for improved antibody effector function. In the NLR+TLD-MTX group, whether allo-B cells exhibit stunted activation, GC formation and other reduced development when compared to transfusion alone will be further examined. Also, primary transfusion for acute responses and multiple transfusions for long-term responses will be examined.

It was hypothesized that TLD-MTX induces an immunotolerant environment in which platelet antigens are introduced during transfusion, leading to the generation of B_{Regs} and T_{Regs} that are donor-specific, alloreactive, and actively suppresses alloimmunity during subsequent platelet transfusions or transplants. B_{Regs} are critical for regulating unwanted humoral responses in autoimmune diseases and GVHD. Understanding the B_{Reg} response to TLD-MTX is critical for determining the long-term benefits of tolerance induction. Measuring alloantibodies alone is insufficient to characterize tolerance, as low alloantibody levels might still be accompanied by memory B cells capable of rapidly differentiating into alloantibody-secreting plasma cells. The presence of alloreactive B_{Regs} would suggest suppression of these responses. Detecting rare allo-B cell populations and differentiating subtle shifts in rare subsets like B_{Regs} from the bulk B cell population poses a challenge. To address this, an experimental strategy with MHC tetramers to identify allo-B cells by flow cytometry was optimized. MHC tetramers have been effectively used to examine allo-B cells in cardiac transplant models and this strategy was successfully employed in the platelet alloimmunization model to identify allo-B cells specific for class I and class II MHC alleles. Robust T cell-dependent B cell activation requires help from CD4⁺ T cells. Conversely, T_{Regs} can regulate and suppress allo-B cell responses. Although TLD-MTX ITI is believed to be mediated by B_{Regs}, T_{Regs} are hypothesized to contribute and thus T cells will be examined for activation, cytokine production, and induction of regulatory T follicular helper cells, and T_{Regs}.

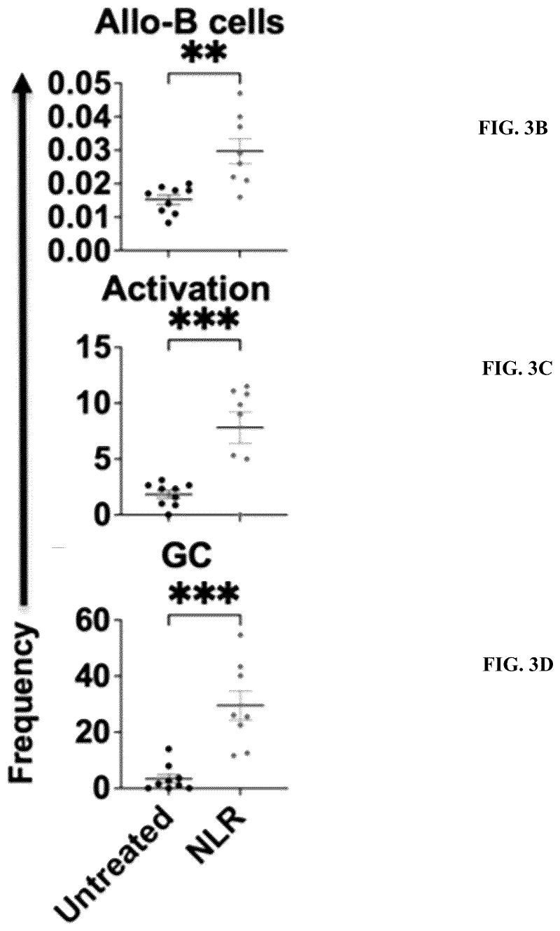

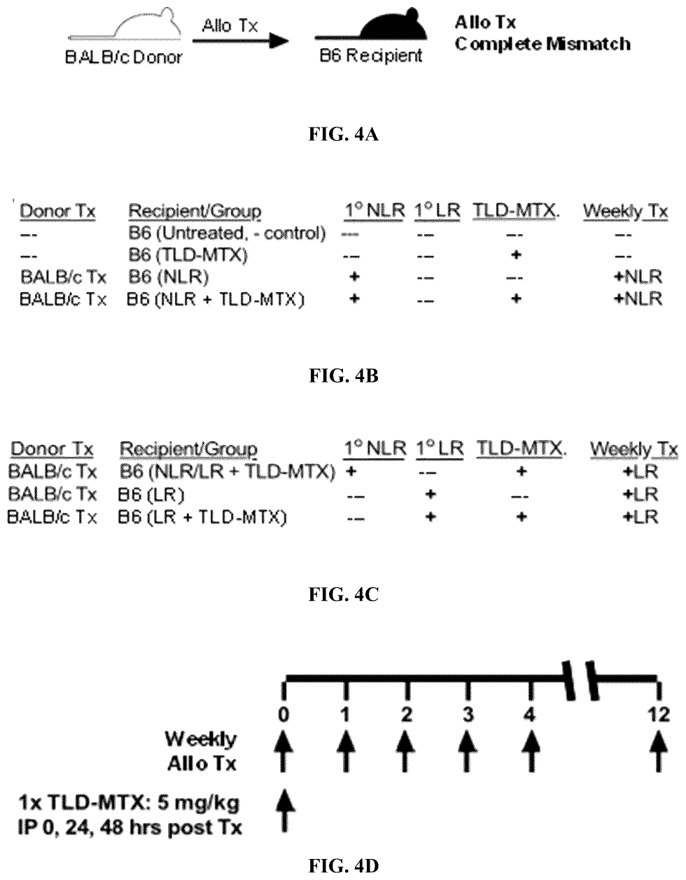

Studies will be conducted in mice. Testing in mice provides additional benefits that would otherwise be challenging. The donor and recipient alloantigen type can be controlled to model alloimmunization, the genetics of established strains are known, and a wide array of tools are available like MHC tetramers. The induction of allogeneic MHC-specific regulatory B cells by TLD-MTX will be investigated. Recipient C57B1/6 (B6) mice will receive TLD-MTX with and without allogeneic platelets from BALB/c donors for the very first transfusion (FIGS. 4A-4D). Some mice will receive weekly allogeneic transfusions for a duration of 12 weeks (FIG. 4B and FIG. 4D). Control groups will include untreated and TLD-MTX only mice. A cohort will be assessed 14 days after the initial transfusion to evaluate acute B cell responses. Another cohort will be examined 10 days following the 12^{th} transfusion to assess long-term B cell responses after multiple transfusions. Acute responses will be evaluated by collecting spleen and lymph nodes, while bone marrow samples will be evaluated for long-term B cell responses such as plasmablasts and long-lived plasma cells. Samples will be split in half to analyze alloreactive B cells specific for class I and class II MHC using H-2D^{d} and I-A^{d} MHC tetramers, respectively. B cells will be stained to examine for activation, GC formation, and memory. B_{Regs} will be examined and if B_{Regs}are induced, they will be confirmed by IL-10 expression. Preliminary staining of B10 B_{Regs} by the co-expression of CD1d and CD5 identified a small subset of B_{Regs} within the allo-B cell population that expanded 10 days after an allogeneic transfusion likely due to homeostatic regulation (FIG. 5). A portion of the spleen will be fixed and frozen for immunohistochemistry (IHC) while the remaining spleen will be used for flow cytometry staining.

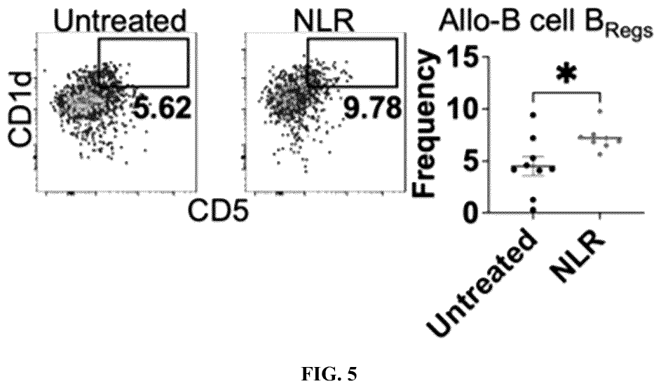

Investigation of the induction of allogeneic antigen-specific regulatory T cells by TLD-MTX will be further performed. Mice will be treated as shown in FIG. 4B. A cohort of mice will be assessed 7 days after the initial transfusion to evaluate acute T cell responses, and another cohort will be assessed 4 days following the 12^{th} transfusion to evaluate long-term T cell responses. For T cells, only spleens and lymph nodes will be evaluated. Lymph nodes will be examined for bulk and alloreactive donor-specific CD4⁺ T cell (allo-T cell) responses. Splenic samples will be split into three parts to analyze bulk T cell responses, intracellular cytokine staining, and allo-T cell responses. Allo-T cells will be examined using class II MHC:2W1S tetramers after magnetic enrichment. The 2W1S peptide is derived from the a chain of the I-E^{d}MHCII molecule (BALB/c mice are of the I-E^{d} haplotype). It has been previously shown that staining with the class II MHC:2W1S tetramer showed a rare but reproducible population of antigen-inexperienced (CD44⁻) 2W1S-specific CD4⁺ T cells in untreated B6 mice, and these allo-T cells robustly expand 7 days after a subcutaneous injection of 2W1S peptide emulsified in adjuvant. T cells will be stained to assess activation, antigen-experience, anergy, T_{FH}, and the induction of T_{Regs}. Intracellular cytokine staining of bulk T cell responses for IFN-γ, TGF-β, IL-4, IL-5, IL-10, and IL-13 will be conducted after stimulation with PMA/ionomycin. The response of T_{Regs} to an allogeneic transfusion is shown in FIG. 6A-6B. Additionally, a portion of the spleen will be fixed and frozen for IHC.

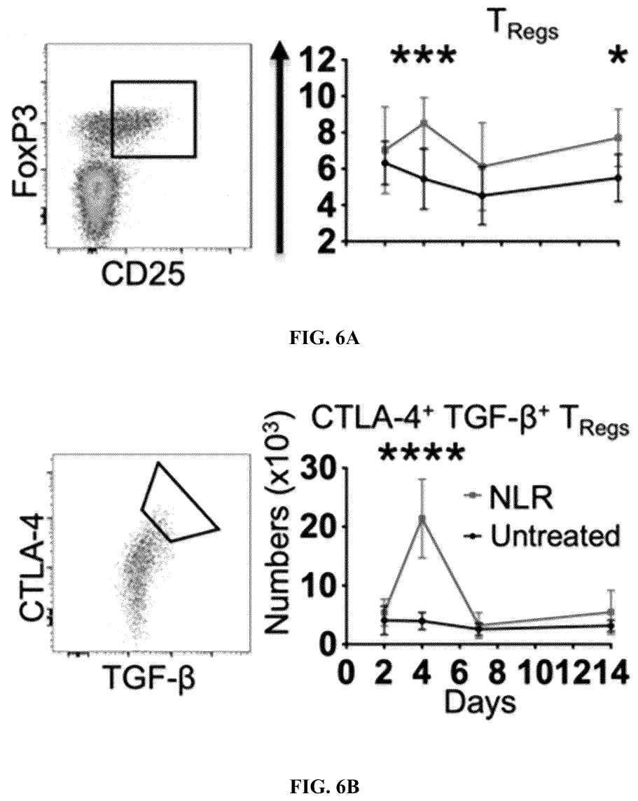

The induction of an immunotolerant cytokine milieu by TLD-MTX will be determined. The same groups as described in FIG. 4B will be evaluated for early cytokine levels by examining serum. Blood will be collected at hours 0, 4, 24, and 48 after transfusion and screened for cytokines by multiplexing methods. Cytokine levels examined after transfusion in serum and in supernatants of mixed lymphocyte reactions is shown in FIG. 7.

### Methods

Mice: B6 and BALB/c mice were purchased from The Jackson Laboratory and acclimated two weeks prior to use. Recipient B6 mice were 9 to 11 weeks old, while BALB/c donors were 2-6 months old. Approximately, an equal number of male and female donors were used for transfusions and as recipients for each experiment. Additional experiments were conducted if differences in sex were observed. Mice are bred and maintained in a specific pathogen-free vivarium at VRI under Animal Welfare Assurance A3367-01, with oversight by the Institutional Animal Care and Use Committee at Covance Laboratories, Inc. (San Carlos, CA).

Immune Tolerance Induction: Recipient B6 mice were given TLD-MTX concurrently with the first platelet transfusion. A single cycle of TLD-MTX consists of three consecutive daily ip doses of methotrexate at 5 mg per kg of mouse weight. The first TLD-MTX dose was given within 15 minutes of the first transfusion.

Allogeneic Transfusion: NLR platelet products were prepared from pooled whole blood and collected into 14% CPDA-1, isolated by gentle centrifugation and Ficoll separation, with WBCs added back to the platelet product, so the product was WBC enriched. NLR platelets were used to model a worse-case allogeneic transfusion scenario and to elicit robust, detectable responses. Leukoreduced (LR) platelets are prepared as above after collected whole blood was passed through a leukoreduction filter. All platelet products were screened using a hematology analyzer to determine platelet and WBC concentrations. Typical concentrations are 4×10⁸ platelets/mL and 5×10⁶ WBCs/mL. Platelets were kept at room temperature and transfused within 4 hours of blood collection. Recipient mice received 100 μL by lateral tail intravenous (iv) injection. Mice received a single allogeneic transfusion or weekly transfusions to model chronic transfusion.

Sample preparation: Processing of spleens, inguinal lymph nodes, and bone marrow involved a combination of homogenization by enzymatic digestion, filter passage, RBC lysis, and cell counting. Sample processing included FcR block and inclusion of a viability marker before staining.

Alloreactive B cells: Allo-B cells were identified by class I and class II MHC tetramers (FIG. 3A). A syngeneic decoy (recipient-type MHC) was used to exclude B cells specific for the non-MHC portion of the tetramer. The one MHC-two fluorophore strategy was employed where tetramers with shared MHC specificity were in two different colors (PE and APC), enabling selection of double-positive events (allo-B cells) and exclusion of single-positive cells specific for fluorochrome (non-MHC). Peptides for class I MHC tetramers known to form stable tetramers were selected and have retained the CLIP peptide for class II MHC tetramers. B6 and BALB/c MHC haplotype tetramers were provided by the NIH Tetramer Core Facility. B cells were gated from singlet, live lymphocytes. A lineage cocktail (CD3, CD11b, CD11c, F4/80, and Nkp46) excluded non-B cell events. The B cell antibody cocktail includes markers for pan B cells (B220, CD19), activated (CD86⁺), antigen-experienced (IgD⁻), GC (CD38⁻/CD95⁺), memory (GC⁻/IgD⁻) plasma cells and plasmablasts (IgD⁻/non-B⁻/Sca-1⁺/CD138⁺/B220^{-/+}), and B10 B_{Regs} (CD5⁺/CD1d⁺). If B_{Regs} are elevated, these cells were confirmed by intracellular IL-10. The MHC tetramer staining is shown in FIGS. 3A-3D and FIG. 5.

Alloreactive T cells: Bulk and alloreactive T cells will be evaluated. Alloreactive T cells will be identified by class II MHC:2W1S tetramer of a similar MHC haplotype as the recipient and isolated by magnetic enrichment by microbeads against the fluorophore. The 2W1S peptide will be derived from the donor BALB/c a chain of the I-E^{d} MHCII molecule. T cells was gated from singlet, live lymphocytes. For T cells, a separate lineage cocktail (B220, CD11b, CD11c, F4/80, GR-1 and Nkp46) excludes non-T cell events. The T cell antibody cocktail includes T cell markers (CD3, CD4, and CD8) and phenotypic T cell markers for activation (CD69), antigen-experience (CD44), and anergy (FR4⁺/CD73⁺). Intracellular staining identifies T_{FH} (CXCR5⁺/PD-1⁺/BCL-6⁺) and T_{Regs} (Foxp3⁺/CD25⁺ and/or CTLA- 4⁺/TGF-β⁺), and after 3-hour stimulation with PMA/ionomycin and brefeldin A identifies immunotolerant (TGF-0 and IL-10), T helper 1 (TH1, IFN-γ), and TH2 (IL-4, IL-5, and IL-13) factors.

Immunohistochemistry: For immunohistochemistry, portions of the spleen were fixed in 4% paraformaldehyde at room temperature overnight, then infused with 30% sucrose at 4° C. overnight, and frozen in OCT medium at −80° C. Splenic sections were mounted, stained, and examined for co-localization of B cells (B220⁺) with T_{FH} (CXCR5⁺) cells or T_{Regs} (FoxP3⁺) in extrafollicular regions or GC. MHC tetramers, CD4⁺ T cells, and peanut agglutinin (PNA) were assessed as a GC marker to measure its number, size, and structure.

Cytokine detection: Serum cytokines were measured using a magnetic bead kit (Millipore) on the Luminex 100 platform (Luminex) with BioManager Software (BioRad) for analysis. IFN-7, IL-2, IL-4, IL-5, IL-6, IL-10, IL-12(p40), IL-12(p70), IL-13, IL-17, IL-21, IL-23, and TNF-α are screened.

BALB/c donor platelets were transfused into B6 recipients to achieve a complete allogeneic mismatch, with and without TLD-MTX (FIG. 4A-4B). Mice receiving no transfusion or TLD-MTX alone served as controls. Weekly allogeneic platelet transfusions were administered to model chronic transfusion. Flow cytometry analysis gating was on singlet, live lymphocytes for B cells and T cells. For the analysis of allo-B cells, gating was on B cells, decoy tetramer-negative cells, and then tetramer double-positive events (FIG. 3A). Allo-B cells were phenotyped. Responses in the untreated and TLD-MTX-only treated controls were observed. An early induction of B_{Regs} that actively suppress conventional allo-B cell responses in the NLR+TLD-MTX group, with these B_{Regs} becoming more pronounced after 12 weekly transfusions is expected to be observed. Conversely, platelet transfusion alone is expected to show elevated activation, antigen-experience, and GC formation during early responses, along with increased memory B cells in the spleen and more long-lived plasma cells in the bone marrow after multiple transfusions. The NLR+TLD-MTX group, however, should exhibit suppression of these responses, demonstrating overall dampened B cell alloimmunity.

The groups will be further evaluated for T cell responses. In bulk T cell responses, it was found that a single allogeneic platelet transfusion increases T cell activation, antigen-experience, and cytokine levels from T cells. These effects are expected to persist after multiple transfusions in the platelet transfusion alone group, along with increased TFH cell activity. In contrast, these responses are expected to be broadly suppressed in the NLR+TLD-MTX group. These findings would be further confirmed with IHC where reduced co-localization of B cells and T_{FH} would be observed. T cell tolerance could be mediated by several mechanisms, including active suppression by T_{Regs} and deletion or anergy of alloreactive T cells. These parameters will be examined in the bulk T cell response, with the expectation that FoxP3⁺/CD25⁺/CD4⁺ T_{Regs} are enriched, along with the expression of IL-10 and TGF-β. However, these responses are rare and may be masked within the total T cell population. Therefore, class II MHC tetramers and magnetic enrichment will be used to improve resolution of these allo-T cells. This approach clearly identifies the expansion of T_{Regs} in the NLR+TLD-MTX group in acute responses and after multiple transfusions.

Significant shifts in early cytokine levels associated with transfusion alone is expected, with more subtle shifts associated with TLD-MTX alone. However, dampened cytokine levels in the NLR+TLD-MTX group is expected. Tolerogenic cytokines are expected to be overtly expressed at this stage, but cytokine levels may skew towards a tolerogenic environment and reveal clues regarding the mechanism of tolerance.

If TLD-MTX potentiates B_{Reg} and T_{Reg} induction, then whether cell transfer confers tolerance will be examined. Examining rare T_{Regs} is challenging due to its low frequency in which responses may be masked by the overall T cell population. Class II MHC:2W1S tetramers are used to circumvent this and examine T_{Regs} in the alloreactive population. However, this strategy is limited in that resolution is improved for T cells specific to a single representative donor MHC:peptide, and responses of other TCR specificities is untested. The 2W1S peptide is derived from donor class II MHC and may not reflect responses to donor class I MHC. An alternative strategy to examine antigen-specific tolerance induction is to conduct mixed lymphocyte reactions where donor class I MHC or donor class II MHC feeder cells are co-cultured with recipient splenocytes. The cytokine levels are measured by multiplexing methods in the supernatant (FIG. 7). Examining alloreactive T cells with tetramers will only capture indirect presentation of class II MHC and not account for direct presentation.

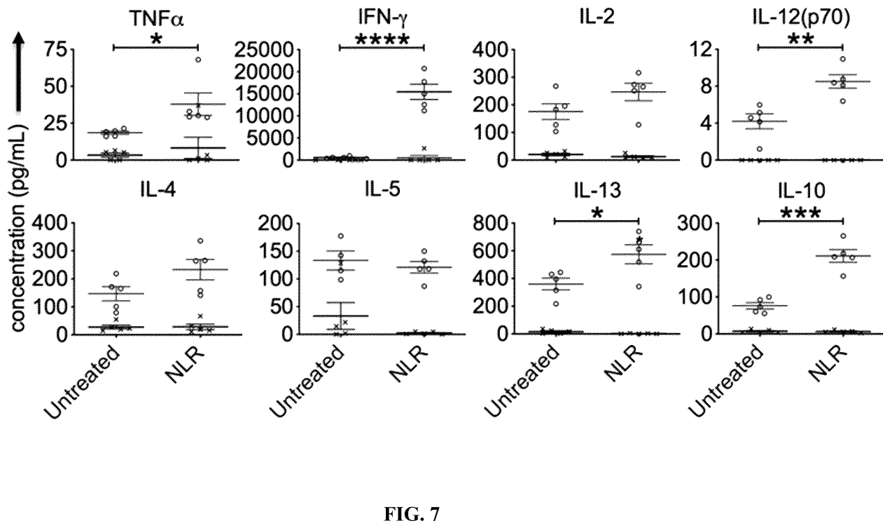

For assessment of cellular responses, the model primarily uses NLR platelets for transfusion to induce more robust all responses and to model a worse-case transfusion. Prior work has shown that primary immune responses to leukoreduced (LR) platelets are similar in type but weaker (FIG. 8) so more challenging to measure. Although repeat LR platelet transfusions eventually achieve similar alloantibody responses as NLR, the use of repeat LR platelet transfusion is not feasible to examine primary cellular responses because responses to repeat LR platelet transfusions differ kinetically and phenotypically. However, alloimmune responses is compared between NLR and LR platelet transfusions. If alloantibody responses differ, key findings in the cellular responses with LR platelets will be confirmed.

Primary B cell responses are expected to peak between 8-14 days and primary T cell responses around day 7 after a single platelet transfusion. However, the peak response may vary for early T cell activation signals or smaller allo-B cell populations. To address this, additional early time points will be examined to determine the kinetics of these responses.

An alloantigen challenge administered through the iv route is expected to elicit a systemic immune response where the primary site is the spleen. However, other lymph node sites may be important to account for migrating immune cells. In a preliminary assessment, responses in the lymph nodes to an allogeneic platelet transfusion in the inguinal, axial, brachial, mesenteric, and cervical lymph nodes were examined and no differences were found. An assessment will be repeated in the context of TLD-MTX.

Based on the optimization studies, H-2D^{d} and I-A^{d} as target class I and class II MHC antigens, respectively, were chosen as targets, as combining tetramers did not improve signal. For the B6 recipients of BALB/c donor platelets, the allogeneic donor MHC alleles are H-2D^{d}, H-2K^{d}, H-2L^{d}, I-A^{d}, and I-E^{d}. H-2D^{d} >H-2K^{d} >H-2L^{d} was found for class I MHC and similar responses between class II alleles (I-A^{d} and I-E^{d}). If the signal with the chosen tetramers is not optimal with TLD-MTX administration, the other alleles are to be re-evaluated.

Evaluating serum, hours after an allogeneic transfusion is the optimal window to observe changes in cytokine levels. However, if these timepoints are too early to examine relevant changes in tolerance induction, cytokines will be examined from the supernatant of co-cultured donor and recipient splenocytes in mixed lymphocyte reactions seven days after transfusion (FIG. 7).

### Example 3: Effect of TLD-MTX on All response to Multiple Platelet Transfusions

It is hypothesized that whether given concurrently with initial transfusion or during the presence of pre-existing alloantibody, TLD-MTX will diminish total alloantibody responses over time and skew isotype responses towards IgG1 with LR and NLR transfusions.

Data demonstrated that TLD-MTX diminished alloantibodies to primary NLR platelet transfusions and clearly showed a reduced trend in the secondary challenge 12 weeks later, where recall responses were skewed towards IgG1 (FIG. 1 and FIG. 2). FIG. 8 shows that the primary immune responses to LR platelets were similar in type to NLR platelets but weaker, and that repeat LR platelet transfusions would eventually achieve similar alloantibody responses as NLR. Similarly, TLD-MTX induction of tolerance to LR platelets will be tested. This is planned by using groups to test LR platelet transfusion with and without TLD-MTX (+/−TLD-MTX, FIGS. 4B-4C). TLD-MTX ITI is most effective when presented with a robust alloantigen, as seen with NLR platelets, allowing recipient immunity to recognize the target antigens to build tolerance against. LR platelets, lacking WBCs, may not be sufficiently antigenic to “train” tolerance induction. This will be evaluated by administering TLD-MTX with a LR platelet transfusion in one group and with a NLR platelet transfusion in another in the initial transfusion, followed by weekly LR platelet transfusions. Although data showed a reduced trend in alloantibodies in the NLR+TLD-MTX group after a secondary challenge at 12 weeks, the durability of this tolerance induction was expected to be longer. This will be tested with a schedule of 12 consecutive weekly transfusions to determine whether tolerance persists in chronically transfused recipients. To test whether TLD-MTX ITI is antigen-specific, a third-party platelet transfusion from an unrelated mouse strain (FVB mice) will be administered during the course of the weekly transfusions and screened for alloantibodies. Given that the standard administration of TLD-MTX is concurrently with the first antigen exposure, its effect in the presence of pre-existing alloantibodies is unknown. It is hypothesized that TLD-MTX will provide benefit in reducing alloantibodies despite the established alloimmunity. This will be tested by administering TLD-MTX during the course of chronic platelet transfusions. It is hypothesized that TLD-MTX will not only diminish total alloantibodies but also skew the isotype towards regulatory IgG1.

To determine if TLD-MTX diminishes alloantibody responses to multiple platelet transfusions, blood serum was collected weekly from the mice after each of the 12 weekly transfusions (FIG. 4B and FIG. 4D). Additional groups include mice given LR platelet transfusions +/−TLD-MTX (FIG. 4C). Another group receives TLD-MTX concurrently with NLR platelets at the initial dose, followed by weekly transfusions with LR platelets. To test for antigen-specific responses, NLR platelets from a third-party mouse strain (FVB) will be introduced six weeks after the initial challenge. Total and isotype alloantibody responses is evaluated using the established class I and class II MHC alloantibody screen by flow cytometry.

To evaluate TLD-MTX on reducing alloantibody responses despite pre-existing alloantibody, a separate cohort of mice will be treated as described above with one change-TLD-MTX will not be administered until the fourth week of the weekly transfusions, at which point TLD-MTX is given concurrently with the 4^{th} weekly transfusion. The respective groups will receive weekly platelet transfusions for another four weeks. Serum will be collected weekly throughout the experiment and screened for alloantibodies.

### Methods

Mice: FVB mice will be used as third-party alloantigens. C.129P2(B6)-β2m^{tm1Unc}/J (BALB/c 02-microglobulin KO) mice will be used to cultivate bone marrow-derived dendritic cells that lack class I MHC antigen, which are essential for our class II MHC alloantibody screening assay.

Allogeneic Transfusion: NLR platelets will be prepared as described above. LR platelets will be prepared just as NLR platelets except whole blood is passed through a Pall leukoreduction filter prior to processing. Typical concentrations for LR platelets are 4×10⁸ platelets/mL and low or undetectable levels of WBCs.

Sample preparation: Serum for alloantibody screens is processed from blood and frozen at −80° C. For terminal bleeds, blood is collected via orbital enucleation. For non-terminal bleeds, small blood volumes are collected from a tail tip incision. Blood is allowed to clot for 20-30 minutes before centrifugation and serum collection.

Alloantibody Screen: Total and isotype alloantibodies from recipient serum is screened against target cells from donor-type mice using an established flow-based method. Serum samples will be assessed against cells expressing only donor-type class I or expressing only donor-type class II MHC molecules. Secondary anti-Igx antibodies (present in approximately 95% of mouse antibodies) stain total serum antibodies, while secondary antibodies targeting IgM, IgG1, IgG2b, IgG2a/c, and IgG3 is used to determine antibody isotype. BALB/c T cells serve as donor-type targets for anti-class I MHC alloantibodies. Cultured bone marrow-derived dendritic cells from β2-microglobulin-deficient BALB/c mice is used as target cells for detecting anti-class II MHC antibodies. FVB T cells serve as donor-type targets for anti-class I MHC antibodies against a third-party alloantigen.

FIG. 8 shows that total alloantibody titers significantly increase after a single NLR allogeneic platelet transfusion and continue to rise with repeated transfusions. LR platelet transfusions elicit a similar, though smaller, alloantibody response as NLR platelets, which also increases with every repeat transfusion. The total and isotype alloantibody responses of allogeneic platelet transfusion +/−TLD-MTX is measured in multiply transfused recipients and recipients with pre-existing alloantibodies.

In the absence of TLD-MTX, total and isotype serum alloantibodies are elevated following an allogeneic NLR or LR platelet transfusion. No alloantibody is detected in untreated or TLD-MTX- only treated groups. When TLD-MTX is administered concurrently with the platelet transfusion, total alloantibody responses is diminished compared to transfusion alone for both NLR and LR groups. Additionally, the isotype skews towards the regulatory IgG1 isotype. This outcome occurs whether TLD-MTX is given concurrently with the first alloantigen exposure or during the presence of pre-existing alloantibodies. Conversely, groups receiving an allogeneic transfusion alone exhibit a more inflammatory alloantibody isotype profile, with elevated IgG2b and IgG2a/c, which have higher effector functions for antibody-dependent cellular cytotoxicity and complement-dependent cytotoxicity. Finally, we expect no difference between groups in responses to a third-party alloantigen.

Alloantibody assay is used to assess effects of various interventions, providing comparative levels of anti-class I and II MHC alloantibodies categorized by isotype. This assay has been dependable, sensitive, and robust, effectively showing subtle shifts in alloantibodies between interventions. If TLD-MTX is not effective with pre-existing antibodies, it may indicate that intervention at 4 weeks is too late to demonstrate benefit. In such a case, TLD-MTX will be tested at the second transfusion to determine if benefit is demonstrated at lower levels of pre-existing alloantibodies.

FVB mice is used as a source of third-party alloantigen, with FVB donor-type T cells utilized to screen class I MHC alloantibodies. If the alloantibody responses to FVB are too small for comparison, or if responses against class II MHCII alloantigen are required, a different third-party mouse strain that has a commercially available β2-microglobulin KO variant, such as C3H/HeJ mice will be used.

### Example 4: Functional Outcomes and Potential Consequences Associated with TLD-MTX

It is hypothesized that TLD-MTX prevents transplant rejection and platelet refractoriness. The effect of TLD-MTX on GVHD in non-leukoreduced platelet transfusions and leukoreduced platelet transfusions will be further examined.

Alloantibodies due to platelet alloimmunization can lead to transplant rejection or target and eliminate subsequent donor platelet transfusions (platelet refractoriness). Lower antibody levels have been associated with reduced antibody-associated sequelae in certain contexts. TLD-MTX has been shown to reduce antibody titers against an enzyme replacement therapy, allowing its continued use for a rare lysosomal storage disease. In a large cohort from the Trial to Reduce Alloimmunization to Platelets (TRAP) study, an assessment of anti-human leukocyte antigen (HLA) antibody levels in platelet recipients revealed that although many participants had anti-HLA antibodies, refractoriness was only associated with the highest antibody levels. Thus, in our preliminary data, while TLD-MTX administration did not eliminate platelet alloantibodies, it reduced them to levels that may have lower functional capacity. This suggests that TLD-MTX could be effective in preventing alloimmune sequelae. To evaluate the functional effects of TLD-MTX, the recovery of transfused platelets or HSCT will be measured to assess platelet refractoriness and transplant rejection in multiply transfused recipients. TLD-MTX, is hypothesized to prevent transplant rejection and platelet refractoriness.

Further, the effect of TLD-MTX on GVHD will be further evaluated. As LR platelet products contain little to no WBCs, GVHD should not pose an issue. However, donor WBCs from NLR platelet products may persist in the recipient. Multiple transfused recipients +/−TLD-MTX will receive platelets from GFP⁺ donors, enabling tracking of platelet recovery to assess the risk of refractoriness. Additionally, leukocytes from GFP⁺ donors will be evaluated to assess the risk of GVHD. To evaluate transplant rejection, multiple transfused recipients will be given HSCT and be assessed for rejection using congenic markers.

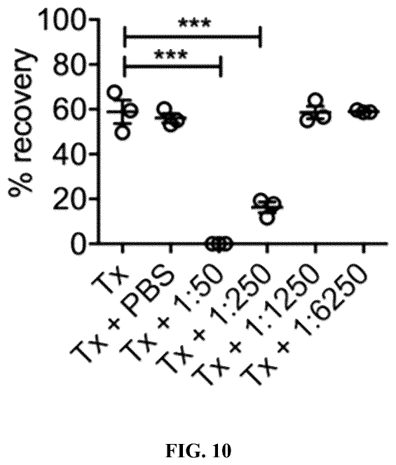

It is hypothesized that TLD-MTX prevents platelet refractoriness in multiply transfused recipients. Groups of mice will be setup as shown FIGS. 4B-4C. After 4 weekly allogeneic platelet transfusions, mice will be given an iv transfusion of GFP⁺ BALB/c NLR platelets. Blood will be collected at 2, 4, 8, and 24 hours post-transfusion to measure levels of circulating GFP⁺ platelets. Spleen and liver will be collected at 24 hours to evaluate platelet clearance. FIG. 10 shows measured platelet refractoriness with this model by the transfer of serum alloantibodies from multiple transfused recipients into naive mice.

It is hypothesized that TLD-MTX modulates GVHD in multiply transfused recipients. Groups of mice will be setup and treated as described above. Mice will be scored daily for clinical signs of disease progression which occurs between 4-7 days. Blood will be collected weekly to measure circulating GFP⁺ leukocytes. Moribund mice, which occurs between 20-30 days, will be euthanized and spleen and liver are collected and evaluated.

It is hypothesized that TLD-MTX protects against the risk of bone marrow transplant rejection. To assess the risk of alloantibodies inducing rejection of HSCT, a separate set of mice will be established as shown in FIGS. 4B-4C. After 4 weekly allogeneic platelet transfusions, the mice will be given antibiotic feed, irradiated, and recipient B6 (congenic CD45.2) will be given HSCT iv from congenic CD45.1 BALB/c donor mice to enable HSCT tracking. Measures of transplant rejection include engraftment kinetics, overall survival, and hematopoietic stem cell/early progenitor numbers. Non-transplanted mice will serve as control.

### Example 5: Safety of TLD-MTX

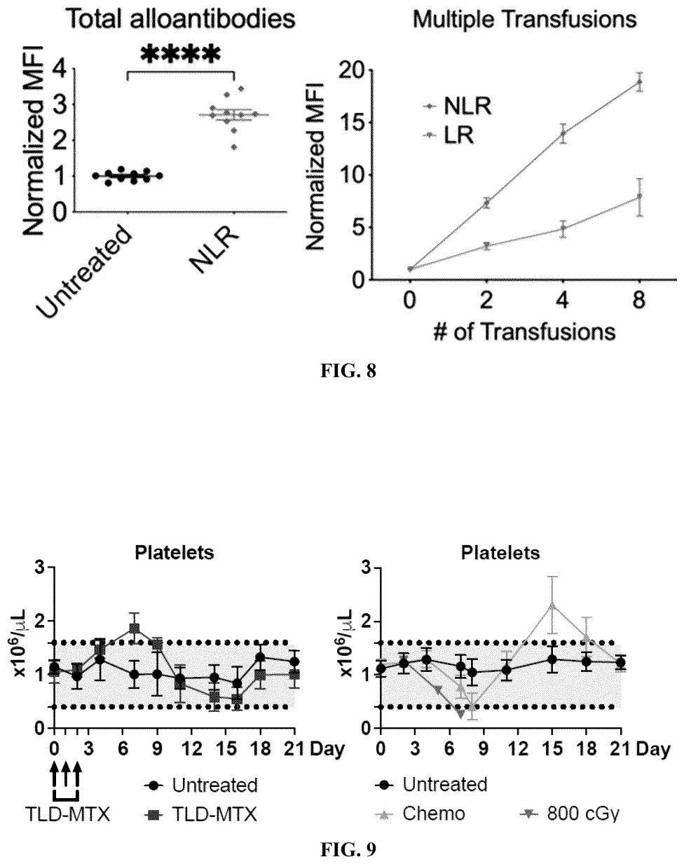

To evaluate the safety of TLD-MTX, the impact of treatment on platelet counts was assessed. Mice were either untreated or given a single cycle of TLD-MTX (5 mg/kg mouse body weight dose daily for 3 days). Mice were monitored for 3 weeks, and complete blood counts were conducted at the indicated timepoints. Safety data (left panel of FIG. 9) demonstrated that TLD-MTX alone does not negatively impact platelet counts in the model. In fact, platelet numbers showed early transient increases before plateau. For comparison, mice treated with chemotherapeutic agents such as cytarabine and doxorubicin or irradiation (800 cGY) (right panel of FIG. 9) induced early thrombocytopenia.

### Methods

Mice: In addition to the mice outlined above, GFP⁺ BALB/c (CByJ.B6-Tg(UBC-GFP)30Scha/J) mice, which express GFP under the human ubiquitin promoter and are homozygous for the H-2^{d} MHC haplotype, will be used as GFP⁺ donors to evaluate platelet rejection and GVHD. BALB/c mice congenic with CD45.1 (CByJ.SJL(B6)-Ptprc^{a}/J) will be used for bone marrow donors and will be transplanted into B6 recipients (CD45.2) for transplant work.

Platelet refractoriness: Groups of mice will be transfused with GFP⁺ BALB/c NLR platelets at the 4^{th} week of weekly platelet transfusions. Blood will be collected at 2, 4, 8, and 24 hours post-transfusion to measure recovery of GFP⁺ platelets. Percent recovery will be determined based on the number of platelets transfused and the estimated total numbers based on weight. GFP⁺ and platelet marker CD41 will identify donor platelets by flow cytometry in recipient blood. Spleen and liver will be collected at 24 hours to evaluate platelet and/or leukocyte clearance. FIG. 10 shows dose-dependent platelet refractoriness in mice using a similar strategy.

GVHD: Groups of mice as described above will be transfused with GFP⁺ BALB/c NLR platelets at the₄₁ week of weekly platelet transfusions. Mice will be scored daily for signs of disease progression (starts 4-7 days). Blood will be collected weekly and evaluated for donor leukocytes with GF^{P}+ and CD45⁺ staining. Moribund mice will be euthanized (20-30 days) and spleen and liver will be evaluated for donor leukocytes.

HSCT transfer: B6 recipient mice will be switched to antibiotic food 5 days prior to irradiation with 850 cGy, followed by iv transplantation with 107 T-depleted bone marrow cells collected from CD45.1 congenic BALB/c donors. Engraftment will be measured at 1, 2, 3, 4, 6, 8, 10, and 12 weeks by collecting a small volume of blood and staining for CD45.1 (donor) and CD45.2 (recipient) for evaluation by flow cytometry.

Evaluation of stem cell rejection: At 12 weeks post-transplant, mice will be injected with 150 mg/kg 5-fluorouracil to kill cycling hematopoietic progenitors and enrich for quiescent stem cells. Bone marrow will be harvested from the femora 24 hours later and counted, with the total bone marrow content estimated based on published values. HPP-CFC assays will be performed as previously described, and expansion cultures will be used to further probe the capacity of the bone marrow samples to grow and generate HPP-CFC. This clarifies the total number of primary HPP-CFC and secondary (culture-expanded) HPP-CFC per mouse. Age-matched, non-transplanted mice will be used as positive controls for these assays.

In multiply transfused recipients without TLD-MTX, high levels of alloantibodies are present which would cause donor BALB/c platelets and WBCs to be rapidly cleared. Conversely, for groups given TLD-MTX, the induction of immune tolerance mechanisms may diminish recipient alloantibody levels and allow donor platelets to survive longer in recipients. Just as alloantibodies are suppressed from rejecting platelets, WBCs also persist longer. Similarly, TLD-MTX prevents rejection of HSCT.

The kinetics of GVHD disease progression and survival is unknown with TLD-MTX. Therefore, the study will examine the optimal timepoints to measure GVHD and earlier and/or later time points are measured as needed. GFP is known to be immunogenic and may pose problems with long-term GVHD. An alternative method would be to use CD45.1 congenic BALB/c mice to enable tracking of donor leukocytes.

### SUMMARY OF EXAMPLES

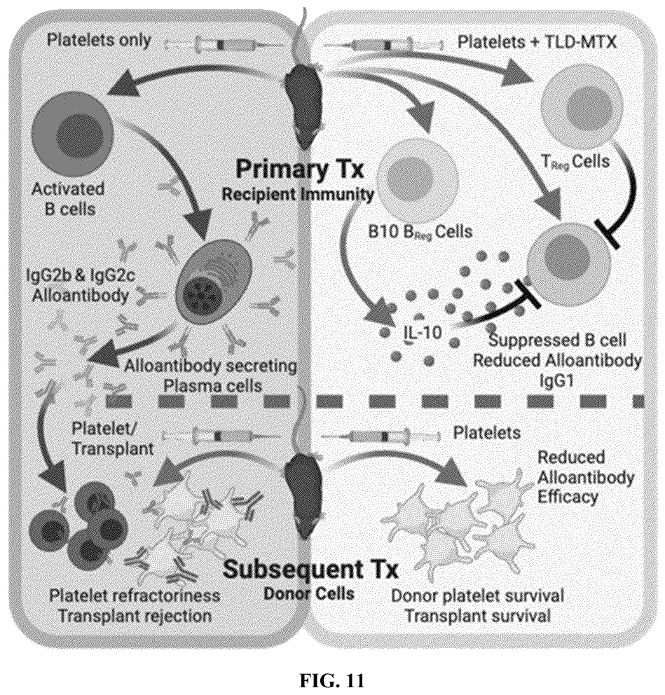

As described herein, a treatment option to diminish alloimmunization to platelet transfusion and prevent alloimmune sequelae, including transplant rejection and platelet refractoriness is provided. Moreover, the examples described herein identify the mechanism of TLD-MTX ITI, as shown in FIG. 11, evaluate the durability of tolerance, and assess the risk of GVHD. Translation of TLD-MTX into the clinic is highly feasible as it is safe, readily available, and addresses an unmet medical need for vulnerable and high-risk populations such as transplant and multiply transfused recipients. The implications of successful TLD-MTX ITI suggest tolerance induction may extend to other antigens, which opens future avenues of research in transplantation and allergy.

What is claimed is:

1. A method of preventing or reducing alloimmunization and/or alloimmune sequelae in a subject in need thereof, comprising:
administering a first dose of methotrexate (MTX) concurrently with a first allogeneic transfusion, thereby preventing or reducing alloimmunization and/or alloimmune sequelae in the subject.

2. The method of claim 1, further comprising administering a second dose of MTX.

3. The method of claim 2, further comprising administering a third dose of MTX.

4. A method of inducing a long-term immune tolerance to prevent or reduce alloimmunization in a subject in need thereof, comprising:
administering a first dose of MTX concurrently with a first allogeneic transfusion;
administering a second dose of MTX; and
administering a third dose of MTX, thereby inducing long-term immune tolerance in the subject.

5. The method of claim 1, wherein the transfusion comprises transfusion of allogeneic platelets.

6. The method of claim 5, wherein the transfusion comprises allogeneic leukoreduced platelets or non-leukoreduced platelets.

7. The method of claim 5, wherein the method prevents or reduces alloimmunization in the subject for at least 12 weeks.

8. The method of any one of the preceding claims, the first, second and third dose of MTX is about 0.1 mg/kg to about 7 mg/kg.

9. The method of claim 1, wherein each of the first, second, and third doses are administered as a daily dose.

10. The method of claim 1, wherein the method in the subject: (a) enhances the level of one or more of regulatory B cells, regulatory T cells, IL-10, and TGF-beta; (b) reduces the level of one or more of TNF-alpha, IFN-gamma, IL-2, IL-12, IL-4, IL-5, and IL-13; and/or (c) reduces the total alloantibody level or skews the alloantibody response towards isotypes that are less inflammatory or more immunotolerant.

11. The method of claim 1, wherein the method prevents or reduces alloimmunization in the subject for a subsequent transfusion.

12. The method of claim 1, further comprising a second transfusion.

13. A method of reducing risk of bone marrow transplant rejection in a subject in need thereof, comprising: administering a first dose of MTX concurrently with a first allogeneic platelet transfusion in a subject planning to undergo or have undergone bone marrow transplant, wherein administering MTX concurrently with platelet transfusion reduces the risk of bone marrow transplant rejection.

14. The method of claim 13, further comprising administering a second dose of MTX.

15. The method of claim 14, further comprising administering a third dose of MTX.

16. The method of claim 15, the first, second, and third dose of MTX is about 0.1 mg/kg to about 7 mg/kg.

17. The method of claim 16, wherein the first, second, and third dose are administered as a daily dose.

18. The method of claim 17, further comprising administering a daily dose of MTX on 4^{th}, 5^{th} , 6^{th}, 7^{th} , 8^{th}, 9^{th}, 10^{th}, or 11^{th} day after administration of the first dose.

19. A method of preventing or reducing alloimmunization and/or alloimmune sequelae in a subject in need thereof, comprising:
administering a first dose of MTX concurrently with a first allogeneic non-leukoreduced platelet transfusion;
administering a second dose of MTX; and
administering a third dose of MTX thereby preventing or reducing alloimmunization and/or alloimmune sequelae in the subject.

20. The method of claim 19, the first, second and third dose of MTX is about 0.1 mg/kg to about 7 mg/kg.

21. The method of claim 19, wherein each of the first, second and third doses are administered as a daily dose.

22. The method of claim 19, wherein the method, in the subject, (a) enhances the level of one or more of regulatory B cells, regulatory T cells, IL-10, and TGF-beta; (b) reduces the level of one or more of TNF-alpha, IFN-gamma, IL-2, IL-12, IL-4, IL-5, and IL-13; and/or(c) reduces the total alloantibody level or skews the alloantibody response towards isotypes that are less inflammatory or more immunotolerant.

23. The method of claim 21, wherein the doses are administered on consecutive days or non-consecutive days.

24. The method of claim 19, the method further comprising administering a dose of MTX before the administration of the first platelet transfusion.
